(19) United States
(12) Patent Application Publication
(10) Pub. No.: US 2015/0157509 A1
(43) Pub. Date: Jun. 11, 2015

(54) OCCLUSIVE SKIN COVERING

(71) Applicants: Oscar Polk Atkinson, Memphis, TN (US); Matthew Singer, Memphis, TN (US); KEVIN YACOUB, Los Olivos, CA (US); MEREDITH POHLAND, Memphis, TN (US)

(72) Inventors: Oscar Polk Atkinson, Memphis, TN (US); Matthew Singer, Memphis, TN (US); KEVIN YACOUB, Los Olivos, CA (US); MEREDITH POHLAND, Memphis, TN (US)

(21) Appl. No.: 14/566,843

(22) Filed: Dec. 11, 2014

Related U.S. Application Data
(60) Provisional application No. 61/914,456, filed on Dec. 11, 2013.

(51) Int. Cl. A61F 13/02; A61F 2/50; A61L 15/24; B29C 65/48; A61L 15/18; A61L 15/52; A61F 13/00; B29C 45/16

(52) U.S. Cl. CPC A61F 13/0256; A61F 13/00063; A61F 2/50; A61F 13/0289; B29C 45/1642; B29C 65/48; A61L 15/18; A61L 15/52; A61L 15/24; A61F 2002/5001; A61F 2310/0097; B29K 2083/005

(57) ABSTRACT

A device for concealing an area of skin is disclosed. The disclosed device can also be used to deliver to the concealed skin area one or more beneficial agents. The device is designed as a laminate having a removable release liner, an occlusive layer, and a finish layer. In some delivery platform embodiments, the occlusive layer includes a reservoir for holding and releasing the one or more beneficial agents to the user's skin. The device is applied to the skin by removing the release liner and placing the exposed adhesive layer of the occlusive device directly on the user's skin.

### CROSS-REFERENCE TO RELATED APPLICATIONS

This application claims the benefit of U.S. Provisional Patent Application No. 61/914,456, filed Dec. 11, 2013, the contents of which are incorporated herein by reference in their entirety.

### FIELD OF THE INVENTION

The present invention relates generally to compositions and methods to conceal a dermatologic blemish, tattoo, wrinkle, or skin damage while also making available the capability of delivering to a scar, discolored or damaged skin a healing or beneficial ingredient.

### BACKGROUND OF THE INVENTION

A consumer seeking to temporarily cover up natural or manmade skin imperfections currently must rely on application of cosmetic compositions to the area to be concealed and the surrounding area. In most cases, heavy cosmetic application must be applied in order to conceal the area. Heavy cosmetic application has many disadvantages, including difficulties related to matching skin color and tone of the skin surrounding or near the blemished or damaged skin to be covered, as well as the relatively high level of skill needed to do this. The heavy wax and pigment of concealing cosmetics is opaque and blocks out the natural skin tone which can create an artificial appearance. The wax can melt in sunny conditions, under stage lighting or other high temperature environments. Traditional concealing cosmetics will smear or wear off with sweat, natural oils from the skin, or contact with other surfaces. Other cosmetic concealment products include setting powders used to reinforce cosmetic coverings and the finishing powders used to minimize surface lines. Color correcting mineral powder foundations are also used to hide discolored skin. These powder based concealment approaches do not conceal the uneven topography of the skin since the powder does not sufficiently fill in deep scars, pock marks or wrinkles.

Consumers seeking to conceal blemished or damaged skin may also benefit from treating the area to be concealed in order to lessen the damage, especially recessed acne and medical scars, as well as age lines and wrinkles. Two chief options currently exist for delivering an active or other beneficial agent to lessen visibility or noticeability of blemished or damaged skin while also concealing the area. The first option is the use of thicken woven coverings, such as gauze pads, medical tape, and/or bandages. This method has the disadvantage of drawing attention to the area being treated, as even “natural” colored bandages tend to have peripheral edges that are visibly perceptible to a user and others. Because the color of the bandage will create an opaque covering of the skin, it is not likely to blend in with the surrounding skin tone. The second option is the use of thinner non-woven coverings made from a variety of materials placed over a layer of applied beneficial agent. However, many such products available today do not blend in with the surrounding skin. They are not textured to match the appearance of the skin nor do these products move and flex naturally with the skin.

There exists a need for a more aesthetically pleasing concealment and delivery platform than the current products and alternatives. There also exists a need for an easier application process for an occlusive covering concealment and delivery platform.

### SUMMARY OF THE INVENTION

Disclosed herein, in one embodiment, is an occlusive device for covering a skin imperfection that includes an occlusive layer having first and second surfaces, a finish layer applied to the occlusive layer, an adhesive layer applied to the occlusive layer, and a removeable release layer adhered to the adhesive layer. The adhesive layer is selected with predetermined adhesive properties to work in concert with the thickness and tensile strength of the release liner such that the adhesive bonds weakly to the release liner to allow it to be removed but sufficiently bonds to skin to retain the occlusive layer on a user's skin until removed by the user. In some aspects, the finish layer is applied to the first surface and the adhesive layer is applied to the second surface. In some aspects, the first and second surfaces of the occlusive layer define a thickness that is constant or variable. The occlusive layer has a central zone and a peripheral zone that in some aspects the thickness of the peripheral zone tapers to an edge and the first surface tapers to the second surface between the central zone and the peripheral zone. In some aspects, the second surface is substantially planar. The central zone of the occlusive layer is 0.010 to 0.40 inches thick and the peripheral zone has an edge thickness of 0.001 to 0.003 inches in some aspects. The first surface of the occlusive layer may be textured to simulate human skin. The occlusive layer may be formed from a variety of materials and in one aspect comprises a polymer composition that may include at least one additive, such as one or more of tackifiers, plasticizers, fillers, antioxidants, stabilizers, pigments, curatives, or crosslinkers. The polymer composition may comprise a thermoset elastomer such as silicone. In some aspects, the occlusive layer has a Shore A hardness of 1 to 20, and may be translucent, colorless and/or waterproof. In some aspects the occlusive layer comprises beneficial agents.

In some aspects, the occlusive layer may further comprise a reservoir that may be an internal space within the occlusive layer, for instance, in the central zone, and the second surface may include an opening that forms a passageway to the internal space of the reservoir. The reservoir may comprise beneficial agents for delivery to the user's skin. The beneficial agents may include nutrients, pharmaceuticals, antimicrobials, or a combination thereof. The central zone of the occlusive layer may include an opening through the central zone that may receive a preformed delivery platform that comprises beneficial agents. The delivery platform may be made from the same material as the occlusive layer, or a different material, and in some aspects may be translucent, and/or coated with a finish layer.

With further reference to the finish layer of the occlusive device, it is absorbent, accepts application of a cosmetic, and is applied by spraying, dipping, or brushing or as a preformed layer. In some aspects the finish layer is about 0.001 (0.025 mm) to about 0.003 inch (0.08 mm) thick and/or has a durometer of from about 70 to about 80 Shore A hardness. The finish layer is also applied to the second surface of the occlusive layer, and in some aspects, is applied between the second surface of the occlusive layer and the adhesive layer. The finish layer in some aspects comprises a polyurethane composition. In certain embodiments the finish layer is a non-adhesive finish layer, while in some embodiments the finish layer comprises an adhesive.

With reference to the adhesive layer, it comprises a pressure-sensitive adhesive, that in some aspects is such that when the occlusive layer is removed from a user's skin without any pain to the user and with minimal adhesive residue on the skin. Alternatively, the adhesive has a peel adhesion force of from about 300 g/cm to about 900 g/cm, or more specifically, from about 500 g/cm to about 700 g/cm. The adhesive layer comprises beneficial ingredients.

With reference to the release liner, it may be transparent, comprises a polymeric material, and/or is made from waxed paper, fluoro-silicone coated polyester, uncoated polyester, polypropylene, polyethylene, or polyolefin.

In some embodiments, the occlusive device further comprises a removable protective layer that, in some instances, is applied to the finish layer. In some aspects the protective layer is translucent. In some aspects, the protective layer is coated with an adhesive that adheres the protective layer to the finish layer. The adhesive is water-soluble and it and the protective layer may be removed by treating with an aqueous solution. Removal of the protective layer leaves the first surface of the occlusive layer without sufficient adhesive residue to interfere with application of cosmetics.

In another embodiment, a method of applying an occlusive device is provided that includes the steps of exposing a second surface having an adhesive layer adhered thereto of an occlusive layer having first and second surfaces, where the first surface of the occlusive layer mimics the appearance of human skin, and adhering the second surface to an application site of skin of a user. In some aspects the step of exposing a second surface having an adhesive layer includes removing a release layer from the occlusive device. The step of exposing a second surface having an adhesive layer may include exposing a reservoir. The step of adhering the second surface may include placing the reservoir on the application site of skin of a user, thereby allowing the delivery of a beneficial agent to the skin of the user. In some aspects, a finish layer is adjacent to the first surface of the occlusive layer. The first surface of the occlusive layer is coated with a finish layer. In some aspects, the method further comprises the step of applying cosmetics to the finish layer. Regarding the occlusive layer, it may include a central zone and a peripheral zone with a thickness defined by the first and second surfaces, and the thickness of the peripheral zone tapers to an edge. In some aspects the first surface tapers to the second surface between the central zone and the peripheral zone.

Another embodiment provides a method of manufacturing an occlusive device. The method comprises providing a polymer composition, injecting the polymer composition into a mold, wherein the mold comprises spaced apart first and second surfaces, wherein the first surface is substantially concave and textured to mimic the appearance of human skin and the second surface is substantially smooth and planar. The method further comprise the step of allowing the polymer composition to cure, wherein the cured polymer composition has physical material properties simulating human skin and has a convex surface and an opposing planar surface. In some aspects the polymer composition comprises at least one additive, such as one or more of tackifiers, plasticizers, fillers, antioxidants, stabilizers, pigments, curatives, or crosslinkers. In some aspects, the method further comprises the steps of applying an adhesive or non-adhesive finish layer over the convex surface. The method may include a step of adhering an adhesive layer to the planar surface, and/or a step of adhering a removeable release liner to the adhesive layer.

### BRIEF DESCRIPTION OF THE DRAWINGS

Further advantages of the invention will become apparent by reference to the detailed description of preferred embodiments when considered in conjunction with the drawings which form a portion of the disclosure and wherein:

FIG. 1 is a top plan view of an occlusive device in accordance with some embodiments of the invention.

FIGS. 2A-2C show in bottom plan view occlusive devices in accordance with some embodiments of the invention.

FIG. 3 shows in cross-section view an occlusive device in accordance with some embodiments of the invention.

FIG. 4 shows in cross-section view an occlusive device comprising a reservoir in accordance with some embodiments of the invention.

FIG. 5 shows a top plan view of the occlusive device of FIG. 4.

FIG. 6 shows in cross-section view an occlusive device comprising a reservoir in accordance with another embodiment of the invention.

FIG. 7 shows a top plan view of the occlusive device of FIG. 6.

FIG. 8 shows in cross-section view an occlusive device comprising a protective layer in accordance with some embodiments of the invention.

FIG. 9 shows in cross-section view an occlusive device comprising a protective layer and reservoir in accordance with some embodiments of the invention.

FIG. 10 shows in cross-section view an occlusive device comprising a protective layer and reservoir in accordance with other embodiments of the invention.

FIG. 11 is a flow diagram showing an illustrative method of manufacturing at least one embodiment the occlusive device presented herein.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Described herein are illustrative embodiments of an occlusive device for concealing skin imperfections. As used herein, “skin imperfections” includes but is not limited to, scars and skin discolorations. Scars include indented or atrophic scars that are recessed below the surface of the surrounding skin. The occlusive device can hide the recessed area by covering the area of uneven skin. Scars also include hypertrophic scars that are raised above the surface of the surrounding skin. Often called keloid scars, these are difficult to conceal because the uneven skin creates a shadow when light is shined on the raised area. The occlusive device can conceal hypertrophic scars by covering the rough, raised skin and gradually blending into the surrounding area of smooth skin. The occlusive device can also conceal contracture scars that are often the result of burns. Flat scars or stretch marks can also be concealed with the occlusive device. “Skin discolorations” as used herein include vitiligo, birthmarks, port wine stains, lupus, age spots, sun spots, freckles, moles, and roseacea, as well as tattoos and similar markings.

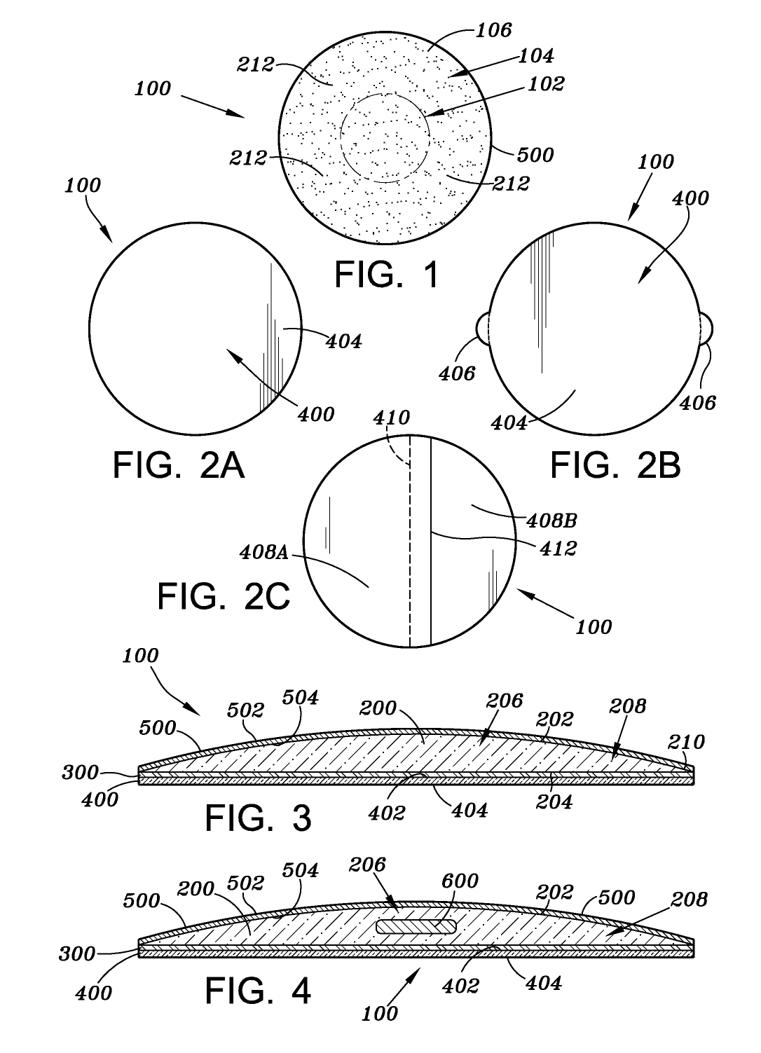

An embodiment of the occlusive device is depicted in FIGS. 1, 2A and 3. As shown in FIGS. 1, occlusive device 100 comprises a central zone 102 and a surrounding peripheral zone 104 that extends to peripheral edge 106. While shown in FIGS. 1 and 2A-2C as approximately circular, occlusive device 100 may comprise a variety of geometric or non-geometric shapes depending on the intended use and application site of the device. FIG. 3 depicts a cross-sectional view of an embodiment of the occlusive device to highlight the laminar construction of the occlusive device 100, which comprises an occlusive layer 200, an adhesive layer 300, and a release liner 400.

Occlusive layer 200 is an internal laminate layer of occlusive device 100. Referring to FIG. 3, the occlusive layer 200 has a first surface 202 and a second surface 204 which define a thickness therebetween, with a central zone 206 and peripheral zone 208 that extends outwardly from the central zone 206 and that extends to a peripheral edge 210. In some embodiments, the first surface 202 extends to meet the second surface 204 at the peripheral edge 210. In some embodiments, the central zone 206 is about 0.010 inch (0.254 mm) to about 0.040 inch (1 mm) thick and more preferably with a thickness of about 0.020 inch (0.51 mm) to about 0.025 inch (0.64 mm). The occlusive layer 200 is preferably molded in thin sheets that mimic the appearance of human skin. It should be understood that the thickness of occlusive layer 200 will vary based on the individual application site characteristics and/or the intended purpose.

In a preferred embodiment, such as illustrated in FIG. 3, the outer edge of the occlusive layer 200 has a tapered geometry, and tapers in thickness at or near the peripheral edge 210 and terminates in an edge thickness of about 0.001 inch (0.03 mm) to about 0.003 inch (0.08 mm) with a preferred thickness of about 0.002 inch (0.51 mm). In some embodiments, a gradual transition in thickness begins at about 0.275 inch (7 mm) from the outside edge to end at a peripheral edge 210 thickness of about 0.001 inch (0.03 mm) to about 0.003 inch (0.08 mm). The peripheral edge 210 permits a visually undetectable transition between the edge of the occlusive device and user's skin that surrounds the application site to achieve the desired aesthetic result. The thicker center allows for concealment of skin imperfections and the thinner edge allow the layer to blend into the surrounding skin.

The second surface 204 is relatively flat or planar, illustrated, for instance, in FIGS. 2A-B, 3, 4, 6 and 8-10, prior to application to a user's skin. As shown in FIGS. 1 and 7, the first surface 202 has a texture 212 and geometry that mimics the texture and appearance of human skin such as the user's that allows the occlusive device to mimic the appearance of human skin. The texture 212 also facilitates the application of cosmetic compositions on the occlusive device by creating micro-pockets for the cosmetic compositions to reside which prevents it from being wiped off. The texture 212 is imparted to the mold by chemical etching, laser etching, or media blasting. The surface roughness (Ra) is preferably about 0.0001 inch (0.003 mm) to about 0.0005 inch (0.013 mm).

The level of opacity of the occlusive layer 200 can also be controlled by the formulation of the material. A more opaque layer will allow for more color concealment in the case of a burn, tattoo, birthmark, or dark scar that is a high contrast from the surrounding skin, or discoloration caused by acne, for example. Occlusive layer 200 can be manufactured in a variety of skin colors and tones to match the user's skin. The color and tone is adjusted by using a combination of pigments, opacifiers, and flocking. Color and tone of the occlusive layer 200 can be adjusted by formulating colors to match the spectrum of human skin tones. Since the covering is translucent, the tone does not have to be an exact match and can be modified with specialty pigments (GSDI, Barberton, Ohio) where the occlusive layer 200 is formulated using liquid silicone rubber. Flocking agents help mimic the natural mottled look caused by the blood vessels in the skin. In a preferred embodiment, colored rayon fibers are used as a flocking agent. The level of translucency is managed by the amount of pigment and opacifiers that are used to tint the composition of the occlusive layer 200. A well formulated color tone will conceal discoloration, but allow the natural skin tone to show through at the thinner edges of the occlusive device.

Alternatively, occlusive layer 200 can be manufactured without the color and tone being adjusted, so that a user's cosmetic or other concealing products is used to mask or blend the occlusive layer into the user's skin color and tone.

Various sizes and shapes may be desired for the occlusive device 100. By way of illustration, where the user's cheek is the site of application of the occlusive device, any shape may be appropriate but it is expected that a preferred dimension is somewhat triangular and would be approximately 6 cm (2.4 inches) in length and 6 cm (2.4 inches) in width. Other shapes from about one-half inch (about 1 cm) to about 6 inches (about 15 cm) or larger may be appropriate for areas of the face, or the body. For example, occlusive device 100 may be relatively elongated to cover a long scar from a mastectomy procedure across any area of the breast or déolletage.

Although occlusive device is depicted as circular in shape, it should be understood that an occlusive device could be any shape, geometric and non-geometric, and still perform the same function of concealment. For example, the occlusive device may be in a variety of shapes such as tear drop, circular, and rectangular.

In another embodiment, the occlusive layer may be provided in universal sizes that can be customized through trimming to meet the exact size required for any given application site.

The occlusive layer 200 may be made from any suitable material, and is preferable made from materials that yield an occlusive layer that exhibits the softness and flexibility of skin. In some aspects, the occlusive layer 200 has a Shore A hardness of 1 to 20, and may be translucent, colorless and/or waterproof. The occlusive layer 200 may comprise a polymer composition, for example, a thermoset elastomer such as silicone. Silicone has been shown to lessen the appearance of scars overtime without the use of agents (see, for example, Gallant-Behm et al., Wound Repair Regen. 2010 March-April;18(2):235-44.). Silicone from Momentive Performance Materials Inc. (Waterford, N.Y.) or Applied Silicone Corporation (Santa Paula, Calif.) may be used. In a further aspect the polymer composition may include at least one additive, such as one or more of tackifiers, plasticizers, fillers, antioxidants, stabilizers, pigments, curatives, or crosslinkers. In some aspects the occlusive layer 200 comprises beneficial agents.

The occlusive device can also be used to deliver beneficial ingredients to the surface of the skin. A beneficial ingredients can be integrated into the occlusive layer 200. Beneficial ingredients may include cosmetic ingredients (e.g., hydroquinone or alpha hydroxyl acids to bleach a tattoo or skin discoloration), nutrients, pharmaceuticals, antimicrobials, and/or other beneficial ingredients known in the skin care field. Further, elastomeric polymer sheeting as an occlusive device has been shown to inhibit evaporation of added beneficial agents, and there are kinetic factors that may increase the absorption of an added agent (see, for example, www.merckmanuals.com/home/skin_disorders/treatment_of_skin_disorders/treatment_of_skin_disorders.html)

To permit application of cosmetic compositions, in some embodiments the occlusive device comprises a finish layer 500 applied to the first surface 202 of the occlusive layer 200, and as illustrated in FIG. 3, finish layer 500 comprises a first surface 502 that faces away from the occlusive layer and a second surface 504 that is adhered or otherwise mated to first surface 202 of occlusive layer 200. The finish layer facilitates the absorption of makeup into the surface of the occlusive device and may be formulated to absorb cosmetic compositions at a similar rate as the user's skin. The finish layer is particularly preferable where the occlusive layer 200 does not allow for the absorption of cosmetic compositions, such as for example where the occlusive layer is made of silicone, which repels liquid and does not allow cosmetic compositions to adhere.

The finish layer can be applied by spraying, dipping, or brushing, or by applying a pre-formed film to the surface, for example, by molding, of the occlusive layer 200. In one embodiment the finish layer comprises a resinous coating. In some embodiments, the composition comprises a silicone composition. Particularly suitable silicone compositions are Momentive SLICK-SIL® or Nusil MED10-6670. In a preferred embodiment, the finish layer has a textured or matte surface that permits deposition of cosmetic compositions that may be formed by the finish layer or by the occlusive layer or both.

The thickness of the finish layer is from about 0.0001 inch (0.003 mm) to about 0.03 inch (0.77 mm), about 0.0001 inch (0.003 mm) to about 0.001 inch (0.03 mm), about 0.0002 inch (0.005 mm) to about 0.001 inch (0.03 mm), and about 0.0003 inch (0.008 mm). In another aspect, the finish layer has a durometer of from about 70 to about 80 Shore A hardness, and preferably no greater than 80 Shore A hardness. The finish layer preferably absorbs liquid makeup. In some embodiments, the finish layer comprises a polyurethane composition.

Referring further to FIG. 3, as well as FIGS. 4, 6, and 8-10, adhered to the second surface is an adhesive layer 300 for adhering the occlusive layer 200 to the user's skin at an application site. The adhesive layer 300 comprises a topical adhesive composition. The adhesive should provide enough tack that the occlusive device adheres to the skin securely but peels away easily with no trauma or pain to the skin and minimal adhesive residue remaining on the skin. In some embodiments, the adhesive has a peel adhesion force of about 300 to about 900 g/cm, and more preferably a peel adhesion force of about 500 to about 700 g/cm. Preferably, the adhesive is hypoallergenic.

In some embodiments, the adhesive layer 300 is pressure-sensitive. The pressure-sensitive adhesive layer has an adhesive force sufficient to adhesively fix the occlusive device to an application site on the skin surface and exhibits an adhesive force that enables the adhesive layer 300 to sufficiently conform to the expansion/contraction of the fabric substrate and to movements of the skin surface. In one embodiment, the pressure-sensitive adhesive layer 300, when subjected to an adhesion test using a bakelite plate as an adherend, exhibits an adhesive force of about 1 to about 7 N/20-mm width, preferably about 2 to about 6 N/20-mm width, and more preferably about 3 to about 5 N/20-mm width.

The pressure-sensitive adhesive which exhibits such an adhesive force is not particularly limited, and use may be made of acrylic pressure-sensitive adhesives, natural-rubber-based pressure-sensitive adhesives, synthetic-rubber-based pressure-sensitive adhesives, silicone type pressure-sensitive adhesives, vinyl ether type pressure-sensitive adhesives, or the like. Of these, acrylic pressure-sensitive adhesives are preferred because the pressure-sensitive adhesive properties thereof are easy to regulate and the acrylic pressure-sensitive adhesives have low skin-irritating properties, etc.

It is preferred that the thickness of the pressure-sensitive adhesive layer be appropriate for the skin application site, etc. However, a thickness, for example, of about 10 to 200 μm, preferably about 20 to 175 μm, and more preferably about 30 to 90 μm. The pressure-sensitive adhesive layer may be disposed on the entire surface of the occlusive layer 200, or may be partly disposed in a pattern, dot, linear, or other suitable arrangement, as long as the arrangement ensures adhesion of the occlusive device to the skin and prevents the peripheral edge(s) 210 from detaching from the application site.

In some embodiments, the beneficial ingredients can also be integrated into the adhesive layer 300 that is in contact with the skin.

Now further referring to FIG. 3, as well as FIGS. 2A-2C, 4, 6, and 8-10, release liner 400 is a removable laminate layer of occlusive device designed to be easily removed or peeled away from occlusive layer 200 prior to application to the user's skin. Release liner 400 protects the adhesive coated second surface of occlusive layer 200. Release liner 400 includes a first surface 402 and a second surface 404. First surface faces toward occlusive layer 200 and the second surface faces away from occlusive layer 200. Release liner 400 can be made of any plastic or polymer material that can be easily removed from the adhesive coated second surface of occlusive layer 200. Materials for the release liner 400 include waxed paper, fluoro-silicone coated polyester, uncoated polyester, polypropylene, polyethylene, or polyolefin.

More preferably, the release liner 400 is made of clear polypropylene to allow the color and tone of the occlusive layer 200 to be visualized before release liner 400 is removed. Release liner 400 may be the same size and shape of occlusive layer 200, or it may have one or more sides or, in addition or alternatively, one or more tabs 406, as illustrated in FIG. 2B, that extend beyond an outer edge(s) of occlusive layer 200 to facilitate removal. An illustrative embodiment with two sides is shown in FIG. 2c, with a first side 408A and a second side 408B, where each side is substantially semicircular and are form two overlapping halves such that a user can take hold of an edge 412 of second side 408B and peel it away, leaving side 408A to be peeled away next such as by grasping edge 410 to expose the adhesive layer 300 for application of the occlusive device 100 to the user's skin.

In one embodiment, the thickness of the release liner 400 is from about 0.0001 inch (0.003 mm) to about 0.08 inch (2 mm). In another embodiment, the thickness of the release liner 400 is from about 0.0003 inch (0.008 mm) to about 0.03 inch (0.8 mm). In another embodiment, the thickness of the release liner 400 is from about 0.001 inch (0.03 mm) to about 0.01 inch (0.3 mm). In one embodiment, the ultimate tensile strength of the release liner 400 is from about 500 (3.45 MPa) to about 60,000 psi (414 MPa). In another embodiment, the ultimate tensile strength of the release liner 400 is from about 1000 psi (6.9 MPa) to about 5000 psi (34.5 MPa). In one embodiment, the protective layer resists tearing on removal from the occlusive device 100.

In certain embodiments, the release liner 400 is a monolayer film or a multilayer film. A monolayer film is a release liner in which a polymer resin is blended with an adhesive. It is noted that the adhesive applied to the release liner 400 would be compatible with adhesive layer 300. Suitable polymer resins from which a polymer film may be manufactured are known in the art, for example, polyethylene, polypropylene, polyvinyl chloride, polyvinylidene chloride, polyester, polyamide, silicone, and blends or copolymers thereof. Polyethylene may be of any suitable type, for example, low density, linear low density, high density, or metallocene. Suitable adhesives include ethylene-acrylic acid, ethylene-vinyl acetate, or mixtures thereof. Methods for blending polymer resins and adhesives are well known in the art using equipment such as high-shear mixers, or single or twin screw extruders. The blended polymer resin and adhesive are then formed into a film by methods known in the art, for example, extrusion, blowing, and casting.

A multilayer film is a release liner 400 to which an adhesive is applied to at least one face prior to application of the release liner to adhesive layer 300. The adhesive may be applied to a polymer film or to a paper sheet, which may optionally comprise a polymer film on the adhesive-coated face, which may optionally comprise a polymer film on the adhesive-coated face. In a preferred embodiment, the release liner 400 is a polymer film. It is noted that the adhesive applied to the release liner 400 would be compatible with adhesive layer 300. Any suitable polymer resin known for forming a polymer film may be used for the polymer film, including, for example, polyethylene, polypropylene, polyvinyl chloride, polyvinylidene chloride, polyester, polyamide, silicone or blends or copolymers thereof. Polyethylene may be of any suitable type, for example, low density, linear low density, high density, or metallocene. In one embodiment, the polymer film may be made a blend of two or more polymer resins. In another embodiment, the polymer film comprises layers of polymer resins.

Preferred adhesive materials for the adhesive component of monolayer films and multilayer films are pressure-sensitive adhesives and hot-melt adhesives that bond weakly to the finish layer on the occlusive device 100. Consequently, the release liner 400 is easily removed from the occlusive layer 200 without leaving any adhesive residue, or otherwise adversely affecting the occlusive layer, for example, by changing the texture, changing the color, or removing a portion of the finish layer. Examples of such adhesive materials include compositions based on polyacrylate, polyvinyl ether, rubber (e. g., natural rubber), polyisoprene, polychloroprene, butyl rubber, neoprene rubber, ethylene propylene diene rubber (EPDM), polyisobutylene, butadiene-acrylonitrile polymer, thermoplastic elastomers, styrene-butadiene rubber, poly-alpha-olefins, amorphous polyolefins, silicones, ethylene-containing copolymers (e.g., ethylene-acrylic acid, ethylene vinyl acetate, ethylene ethyl acrylate, ethylene n-butyl acrylate, and ethylene methyl acrylate), polyurethanes, polyamides, epoxys, polyvinylpyrrolidone and polyvinylpyrrolidone copolymers, polyesters, and mixtures or copolymers thereof. The adhesive component may also contain modifiers, for example, tackifiers, plasticizers, fillers, antioxidants, stabilizers, pigments, curatives, crosslinkers, solvents, and the like.

In another embodiment, the release liner 400 is applied to the bottom or second surface of the occlusive layer 200 by imparting an electrostatic charge to the bottom surface of the release liner 400.

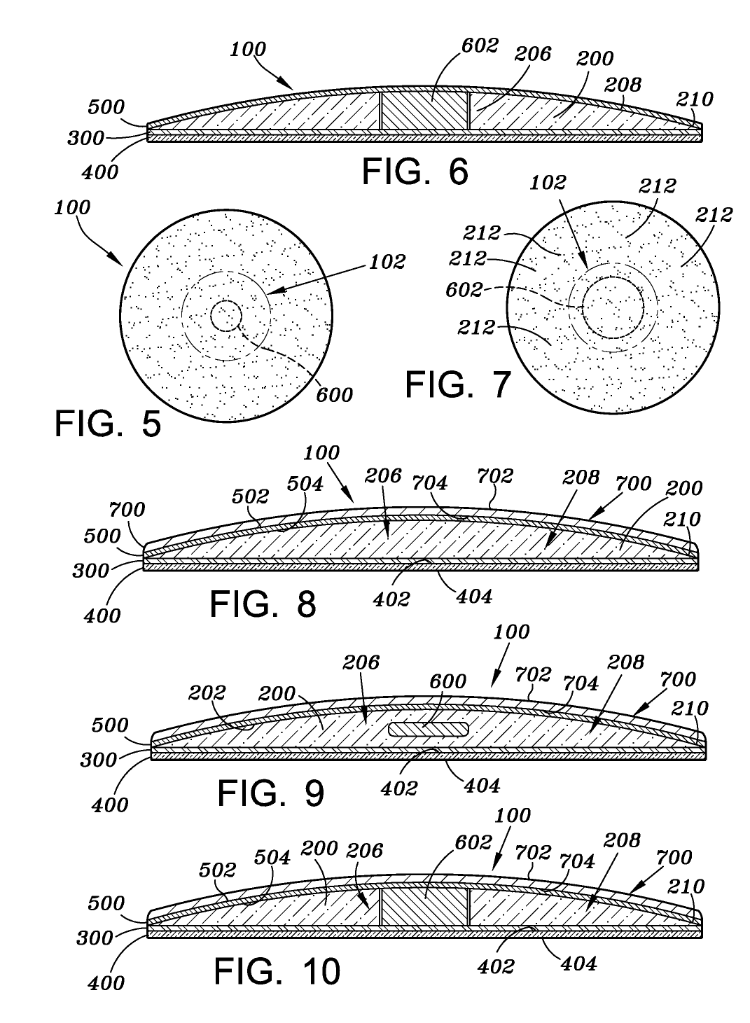

Referring to embodiments illustrated in FIGS. 4-7 and 9-10, occlusive layer 200 further comprises a reservoir 600 or 602 for the delivery of one or more beneficial agents. In some embodiments, reservoir is used as a delivery platform for nutrients or other beneficial ingredients known in the skin care field to promote healthy skin. In other embodiments, reservoir is used as a delivery platform for delivery of beneficial ingredients to promote to promote the improvement in appearance of a skin imperfection.

In some embodiments, reservoir is comprised of occlusive layer 200 or a portion thereof, such that the agent to be delivered is impregnated in the material of occlusive layer or a portion thereof.

In other embodiments, for example as shown in FIGS. 4-7 and 9-10, reservoir 600 is a separate layer or component 602 of occlusive device 100 that can be embedded into occlusive layer 200, attached by adhesive during manufacturing, or applied directly to the skin of a user prior to application of the occlusive device 100. Referring in particular to FIGS. 6-7 and 10, separate delivery platform component 602 is illustrated as a substantially cylindrical piece sized for placement and mating engagement within the central zone 206 of the occlusive layer 200 and maybe secured within the occlusive layer by friction, adhesive, or heating. In these embodiments, the agent to be delivered may be impregnated in the material comprising reservoir or be coated or layered directly onto the material comprising reservoir. In all embodiments, it is fully anticipated that the agent to be delivered could be formulated to be delivered quickly or to be slowly released to the skin of the user. Although not required, preferred embodiments of occlusive device 100 with reservoir are pre-packaged with the nutrients, pharmaceuticals, antimicrobials, and/or other beneficial ingredients.

While reservoir is depicted in the figures as cylindrical in shape, it should be understood that reservoir could be any shape, geometric and non-geometric, and still perform the same function of treatment. It is also noted that reservoir is depicted as a dashed line in the figures, which is done so merely to express that reservoir may not be detectable to the user and other observers. In preferred embodiments, reservoir is visually imperceptible to an observer.

FIGS. 8-10 illustrate embodiments of the occlusive device 100 comprising an optional protective layer 700. Protective layer 700 is a removable laminate layer of occlusive device 100 designed to be removed from occlusive layer 200 only after application of occlusive device 100 to the user's skin. Protective layer 700 helps prevent the peripheral edge(s) 210 from buckling or rolling during application of the occlusive device 100 to the application site on the user's skin. Protective layer 700 includes a first surface 702 and a second surface 704. First surface 702 faces away from occlusive layer 200 and its first surface 202. First surface 702 is the outwardly-facing surface once occlusive device 100 is initially applied to the user's skin. Second surface 704 faces toward the first surface 502 of finish layer 500 and/or layer 202 of the occlusive layer 200. The materials and methods described above regarding embodiments of release liner 400 may also be used for protective layer 700.

Exemplary methods will now be discussed. In one embodiment, a user may apply the occlusive device 100 of the present invention to an application site on the user's skin in order to cover and treat a skin imperfection, for example, a scar. The reason for the need to cover the skin imperfection may affect the method of application of occlusive device 100 and its reservoir. For example, a pre-existing scar on the user's face will determine the extent of the area to be covered by the occlusive device 100. Also factored into the selection of an appropriately sized occlusive device 100 by the user will be the user's skin color and/or tone that will be matched appropriately. After the appropriately sized occlusive device 100 and reservoir have been selected, an appropriate skin color and/or tone that substantially matches the user's skin is selected. It is contemplated that the user may select their tone from a tone chart showing several faces depicting different skin tones. The retail packaging would show the skin tone on the outside of the box and the shapes of the occlusive device 100 inside. If a non-color and tone adjusted occlusive device 100 for a skin imperfection cover up application is chosen, cosmetic compositions may be applied directly to the user's skin or to the occlusive device 100.

It is noted that a user may select an occlusive device 100 having a reservoir prepackaged with a beneficial ingredient. While trimming the occlusive device is possible in certain applications, trimming occlusive device 100 should be discouraged because reservoir could be damaged or compromised, or the seamless effect of provided by the tapered geometry could be lost.

Next, the release liner 400 is removed from the occlusive device 100 to expose the adhesive layer 300 on the second surface of occlusive layer 200. The adhesive layer 300 on the second surface of the occlusive layer 200 is then applied to an application site on the user's skin and the device is held in place with firm pressure for several seconds. Where the occlusive device 100 comprises the optional protective layer 700, after the adhesive layer 300 is adhered to the user's skin at the application site, the protective layer 700 is removed as described above. The result is a visual concealment of the skin imperfection (e.g., scar or tattoo) on the user's skin at the application site.

The same process is followed when a non-color and/or tone adjusted occlusive layer 200 is used except the lack of intrinsic coloring and/or tone adjustment in occlusive layer ordinarily requires further concealment of the skin imperfection by applying cosmetic compositions or a concealing product directly on the user's skin or on the applied occlusive layer to mask the scar from visual observation.

In a still further embodiment, occlusive device 100 may be embedded with one or an array of biosensors to collect information (e.g., pulse, temperature, skin moisture level, etc.) about the user during its application. The biosensors are preferably flexible, so that occlusive device 100 may be placed anywhere on the user's body. The sensor embodiment also includes a power source (e.g., a battery) in electrical communication with the sensor(s). The power source may be single use or reusable (i.e., rechargeable). The sensor embodiment further includes a memory device in electrical communication with the sensor(s) for storing the collected information. The stored information in the memory device may be transmitted to a receiver device via a transmitter device (e.g., an RF transmitter) in electrical connection with the memory device in real time or at a later time. Alternatively, the stored data can be collected after use by removing the memory device and extracting the data by an appropriate device or connecting an extracting device directly to the sensor embodiment.

### Description of Manufacturing Process

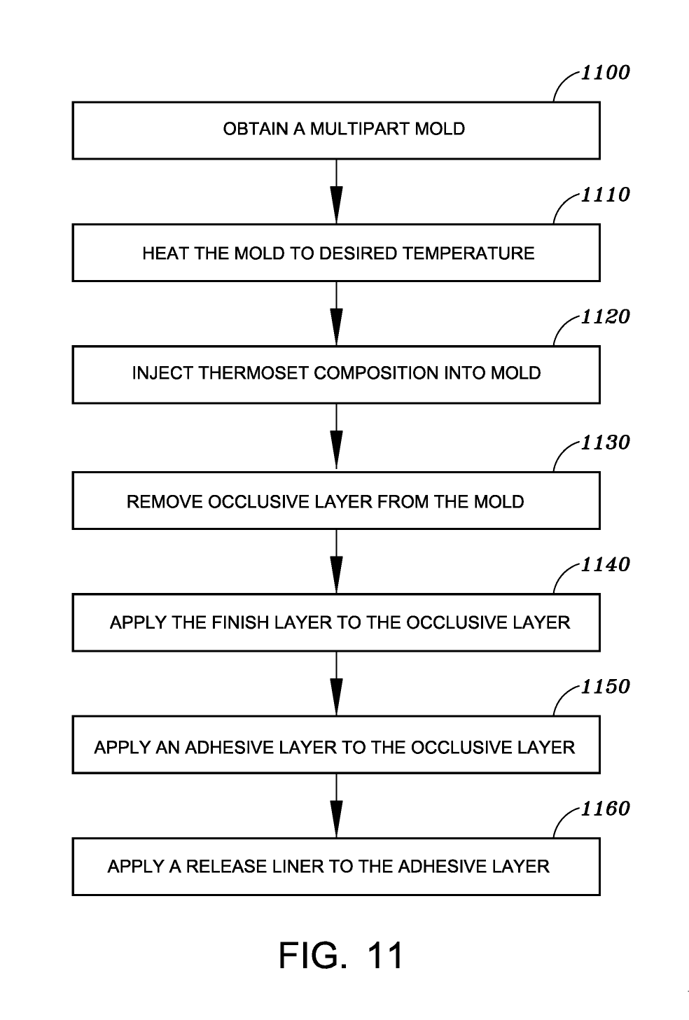

An illustrative method of manufacturing the occlusive device 100 is provided, as shown in FIG. 11. At step 1100, a multipart mold is provided that meets the specifications for the occlusive layer 200, also as described previously. For example, in one embodiment, one surface of the mold corresponding to the first surface 202 of the occlusive layer 200 has a tapered geometry to achieve a thicker center and a thinner periphery and is textured to mimic the appearance of human skin. The other surface of the mold is substantially planar or flat that will form the second surface 204 of the occlusive layer 200.

As described previously, according to a preferred embodiment the first or upper surface of the occlusive device 100 is textured to allow the occlusive device to mimic the appearance of human skin. The texture also facilitates the application of cosmetic compositions on the occlusive device by creating micro-pockets for the cosmetic compositions to reside which prevents it from being wiped off. The texture is imparted to the mold by chemical etching, laser etching, or media blasting. In certain embodiments, the surface roughness (Ra) is about 0.0001 inch to about 0.0005 inch, 0.0002 inch to about 0.0004 inch, about 0.00025 inch to about 0.00035 inch, and about 0.0003 inch.

At step 1110, the mold is next heated to approximately to about 280° F. (138° C.).

At step 1120, a suitable polymer composition for preparing an occlusive layer is provided using an injection molding process. Such compositions have been described previously. The polymer composition in liquid form is injected into the mold. In a preferred embodiment, a two-part platinum-catalyzed silicone is injected into the mold. The polymer composition is allowed to cure from generally 10 to 90 seconds at from about 270° F. (132° C.) to about 400° F. (204° C.), depending on the desired size and configuration for the occlusive layer.

At step 1130, the occlusive layer 200 is removed from the mold and placed on a carrier film with the planar second surface 204 of the occlusive layer on the carrier film and the first surface 202 facing up. The cured occlusive layer 200 has minimal shrinkage or flashing.

At step 1140, the finish layer is applied to the first surface 202 of the occlusive layer 200 to form an occlusive device 100. In a preferred embodiment, the finish layer comprises composition that is preferably applied by spraying. The composition is spray on the first surface 202 of the occlusive layer 200 to form the occlusive device. The finish layer is allowed to dry for between about 10 and 15 minutes at room temperature, or about 1 to about 3 minutes if dried at from about 150° F. (66° C.) to about 250° F. (121° C.). As noted previously, in a preferred embodiment the finish layer comprises a polyurethane composition. In certain embodiments, a film is applied to the non-skin contoured surface of the occlusive layer 200. The film can be laid into the textured surface of the mold so that the first surface 202 of the occlusive layer 200 bonds with the film. In certain embodiments, the film has a thickness of from about 0.001 inch (0.03 mm) to about 0.003 inch (0.08 mm) with a textured or matte finish and a durometer of from about 70 to about 80 Shore A hardness. In some embodiments this layer absorbs liquid makeup. In certain embodiments, the composition comprises polyurethane.

At step 1150, the occlusive device 100 is then turned over with the first surface laying on the carrier film, with the second surface 204 of the occlusive layer 200 facing up. In a preferred embodiment, the adhesive layer 300 is applied to the second surface 204 of occlusive layer 200. And in certain embodiments the adhesive layer 300 is applied by spraying.

Next, at step 1160, the release liner 400 is applied to the adhesive layer 300. In some embodiments, the release liner 400 may be pre-cut to shape and it may be slit down the middle of the part to allow for one half of the liner to be removed at a time.

In optional steps, the occlusive device 100 may be encapsulated into individual paper or foil pouches, and one or more and therefore a plurality of occlusive devices may be packaged into cartons.

### EXAMPLES

### Example 1

After the occlusive layer was ejected from the mold, cosmetics were applied to the first surface of the occlusive layer and tested for adherence by wiping. The cosmetics were smudged and not adequately retained.

### Example 2

An 8% v/v polyurethane solution was applied via spraying the textured first surface of the occlusive layer to create a finish layer of approximately 0.025 inch (0.635 mm) thick. The polymer was allowed to dry for about five minutes. Cosmetics were applied to the finish layer and tested for adherence by wiping but the cosmetics were successfully retained.

### Example 3

An 8% v/v polyurethane solution was applied via spraying the smooth untextured first surface of the occlusive layer to create a finish layer of approximately 0.025 (0.635 mm) inch thick. The polymer was allowed to dry for about five minutes. Cosmetics were applied to the finish layer and tested for adherence by wiping. Cosmetics were not retained when applied to a finish layer covering an occlusive layer having a smooth untextured first surface.

### Example 4

A 3 mil thick polyurethane film was applied via molding the film onto the first surface of the occlusive layer and allowed to cool to form a finish layer of approximately 0.003 inch (0.77 mm) thick to form an occlusive device. The occlusive device was applied to a user's skin and cosmetics were applied to the finish layer. A finish layer of 0.003 inch thickness created a peripheral edge that was visually perceptible to the user and observers, and was therefore deemed to be too thick for an acceptably smooth transition to the skin surrounding the application site.

### Example 5

A 0.003 inch (0.77 mm) thick polyurethane film was applied via molding the film onto the first surface of the occlusive layer and allowed to cool to form a finish layer of approximately 0.003 inch thick. The resulting finish layer had a Shore A hardness greater than 80 that diminished the elasticity of the occlusive device and was therefore undesirable for use.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention.

As used herein, unless otherwise specified or unless the context otherwise clearly requires, “about” regarding a number or measurement means within 10% of the number or measurement.

Whenever a range is disclosed, all subranges and individual values are intended to be encompassed, and when the range refers to integers, every integer from the minimum to the maximum values of such range is included. In addition, where multiple ranges are provided to describe a concentration or characteristic, such ranges may be combined.

This invention is not to be limited by the embodiments disclosed, including any shown in the drawings or exemplified in the specification, which are given by way of example and not of limitation. While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

1. An occlusive device for covering a skin imperfection, wherein the device comprises:
an occlusive layer having first and second surfaces, a non-adhesive finish layer applied to the occlusive layer, an adhesive layer applied to the occlusive layer, and a removable release layer adhered to the adhesive layer, wherein the adhesive layer is select with predetermined adhesive properties to work in concert with the thickness and tensile strength of the release liner such that the adhesive bonds weakly to the release liner to allow it to be removed but sufficiently bonds to skin to retain the occlusive layer on a user's skin until removed by the user.

2. The occlusive device of claim 1, wherein the finish layer is applied to the first surface and the adhesive layer is applied to the second surface.

3. The occlusive device of claim 1, wherein the first and second surfaces of the occlusive layer define a thickness and wherein the thickness is variable.

4. The occlusive device of claim 3, wherein the occlusive layer has a central zone and a peripheral zone and the thickness of the peripheral zone tapers to form a peripheral edge.

5. The occlusive device of claim 4, wherein the second surface is substantially planar and the first surface tapers to the second surface as it extends from the central zone to the peripheral zone.

6. The occlusive device of claim 4, wherein the central zone is from about 0.010 to about 0.40 inch thick.

7. The occlusive device of claim 4, wherein the peripheral zone has an edge thickness of about 0.001 to about 0.003 inch.

8. The occlusive device of claim 1, wherein the first surface is textured.

9. The occlusive device of claim 1, wherein the occlusive layer comprises a polymer composition.

10. The occlusive device of claim 9, wherein the polymer composition comprises one or more of tackifiers, plasticizers, fillers, antioxidants, stabilizers, pigments, curatives, or crosslinkers.

11. The occlusive device of claim 9, wherein the polymer composition comprises a thermoset elastomer.

12. The occlusive device of claim 11, wherein the thermoset elastomer is silicone.

13. The occlusive device of claim 1, wherein the occlusive layer has a Shore A hardness of 1 to 20.

14. The occlusive device of claim 1, wherein the occlusive layer is translucent and waterproof.

15. The occlusive device of claim 1, wherein the occlusive layer comprises beneficial agents.

16. The occlusive device of claim 5, wherein the occlusive layer further comprises a reservoir.

17. The occlusive device of claim 16, wherein the reservoir is an internal space within the central zone and wherein the second surface comprises an opening forming a passageway to the internal space.

18. The occlusive device of claim 17, wherein the reservoir comprises beneficial agents.

19. The occlusive device of claim 18, wherein the central zone comprises an opening for receiving a preformed delivery platform comprising beneficial agents.

20. The occlusive device of claim 19, wherein the delivery platform is made from the same material as the occlusive layer.

21. The occlusive device of claim 19, wherein the delivery platform is translucent.

22. The occlusive device of claim 19, wherein the beneficial agents include nutrients, pharmaceuticals, antimicrobials, or a combination thereof.

23. The occlusive device of claim 1, wherein the finish layer is absorbent.

24. The occlusive device of claim 1, wherein the finish layer is applied by spraying, dipping, or brushing.

25. The occlusive device of claim 1, wherein the finish layer is applied as a preformed layer.

26. The occlusive device of claim 1, wherein the finish layer is about 0.001 to 0.003 inches thick.

27. The occlusive device of claim 1, wherein the finish layer has a durometer of from about 70 to about 80 Shore A hardness.

28. The occlusive device of claim 1, wherein the finish layer comprises a polyurethane composition.

29. The occlusive device of claim 1, wherein the adhesive layer comprises a pressure-sensitive adhesive.

30. The occlusive device of claim 1, wherein the adhesive layer is removed from a user's skin without adhesive residue on the skin.

31. The occlusive device of claim 1, wherein the adhesive has a peel adhesion force of from about 300 to about 900 g/cm.

32. The occlusive device of claim 31, wherein the adhesive has a peel adhesion force of from about 500 to about 700 g/cm.

33. The occlusive device of claim 1, wherein the adhesive layer comprises beneficial ingredients.

34. The occlusive device of claim 1, wherein the release liner is made from a polymeric material.

35. The occlusive device of claim 1, wherein the release liner is made from waxed paper, fluoro-silicone coated polyester, uncoated polyester, polypropylene, polyethylene, or polyolefin.

36. The occlusive device of claim 1, wherein the occlusive device further comprises a removable translucent protective layer.

37. The occlusive device of claim 36, wherein the protective layer is applied to the finish layer.

38. The occlusive device of claim 37, wherein the protective layer is coated with an adhesive that adheres the protective layer to the finish layer.

39. The occlusive device of claim 38, wherein the adhesive is water-soluble.

40. The occlusive device of claim 39, wherein the protective layer is removed by treating with an aqueous solution.

41. The occlusive device of claim 40, wherein removal of the protective layer leaves the first surface of the occlusive layer without sufficient adhesive residue to interfere with application of cosmetic compositions.

42. A method of applying an occlusive device comprising the steps of:
exposing a second surface having an adhesive layer adhered thereto of an occlusive layer having first and second surfaces, wherein the first and second surfaces define a thickness, and wherein the first surface of the occlusive layer mimics the appearance of human skin; and
adhering the second surface to an application site of skin of a user.

43. The method of claim 42, wherein the step of exposing a second surface having an adhesive layer includes removing a release layer from the occlusive device.

44. The method of claim 42, wherein the step of exposing a second surface having an adhesive layer includes exposing a reservoir.

45. The method of claim 42, wherein the step of adhering the second surface includes placing the reservoir on the application site of skin of a user, thereby allowing the delivery of a beneficial agent to the skin of the user.

46. The method of claim 42, wherein a non-adhesive finish layer is adjacent to the first surface of the occlusive layer.

47. The method of claim 46, further comprising the step of applying cosmetic compositions to the finish layer.

48. The method of claim 52, wherein the occlusive layer has a central zone and a peripheral zone and wherein the thickness of the peripheral zone tapers to form a peripheral edge.

49. A method of manufacturing an occlusive device comprising:
providing a polymer composition;
injecting polymer into a mold, wherein the mode comprises spaced apart first and second surfaces, wherein the first surface is substantially concave and textured to mimic the appearance of human skin and the second surface is substantially planar; and
allowing the polymer composition to cure, wherein the cured polymer composition has physical material properties simulating human skin and has a convex surface and an opposing planar surface.

50. The method of claim 49, wherein the polymer composition comprises one or more of tackifiers, plasticizers, fillers, antioxidants, stabilizers, pigments, curatives, or crosslinkers.

51. The method of claim 49, further comprising the step of applying a non-adhesive finish layer over the convex surface.

52. The method of claim 49, further comprising the step of adhering an adhesive layer to the planar surface.

53. The method of claim 49, further comprising the step of adhering a removeable release liner to the adhesive layer.
